(19) United States
(12) Patent Application Publication
(10) Pub. No.: US 2023/0098580 A1
(43) Pub. Date: Mar. 30, 2023

(54) LENS PROVIDING BOTH POSITIVE AND NEGATIVE DIFFRACTION

(71) Applicant: Prakhyat ROOP, Meerut, Uttar Pradesh (IN)

(72) Inventor: Prakhyat ROOP, Meerut, Uttar Pradesh (IN)

(21) Appl. No.: 17/908,257

(22) PCT Filed: Mar. 18, 2021
(86) PCT No.: PCT/IB2021/052263

§ 371 Date: Aug. 31, 2022

(30) Foreign Application Priority Data
Mar. 20, 2020 (IN) 202011012107

(51) Int. Cl. A61F 2/16

(52) U.S. Cl. CPC A61F 2/1618; A61F 2/1654; G02C 7/044

(57) ABSTRACT

A lens device comprises a base lens formed with a plurality of diffractive zones, each having a negative diffractive subzone and a positive diffractive subzone. The negative and the positive diffractive subzones are optionally and preferably arranged alternately. Each diffractive subzone typically has a constant-sign curvature along a radial direction of the base lens.

### RELATED APPLICATION

This application claims the benefit of priority of Indian Patent Application No. 202011012107 filed on Mar. 20, 2020, the contents of which are incorporated herein by reference in their entirety.

### FIELD AND BACKGROUND OF THE INVENTION

The present invention, in some embodiments thereof, relates to optics and, more particularly, but not exclusively, to a lens providing both positive and negative diffraction.

The visual functioning of a person may be impaired due to refractive errors such as near-sightedness, far-sightedness, presbyopia, cataracts and so on. With advent of technology, patients may have more expectations about their vision and may desire spectacle independency. A diffractive-refractive intraocular lens (IOL) is a type of lens which may be planted in the eye as a replacement for eye’s natural lens. There may be different types of IOLs such as monofocal lens, diffractive-refractive multifocal lens, accommodative lens and so on.

The diffractive-refractive IOLs may be designed to comprise a refractive base lens and a diffractive element, superimposed over the base lens. The superimposition of a diffractive element results in concentric zones, called diffractive zones, and are be responsible for diffraction of light. Each of the diffractive zone may be demarcated by steps wherein base of each step may start from the base lens. For light incident at an angle ‘θ’, fraction of light scattered at the step of lens is given by:

$$L = \frac{H Tan ( θ )}{d r}$$

wherein, H is the height of the diffractive step, and dr is the spacing between the diffractive steps.

### SUMMARY OF THE INVENTION

According to an aspect of some embodiments of the invention there is provided a lens device. The lens device comprises a base lens formed with a plurality of diffractive zones, each having a negative diffractive subzone and a positive diffractive subzone. The negative and the positive diffractive subzones are optionally and preferably arranged alternately, wherein each diffractive subzone has a constant-sign curvature along a radial direction of the base lens.

According to some embodiments of the invention at least one diffractive zone comprises a step between a respective negative diffractive subzone and a respective positive diffractive subzone, the step separating a plane where the respective positive diffractive subzone starts from a plane at which the respective negative diffractive subzone ends.

According to an aspect of some embodiments of the present invention there is provided a lens device. The lens device comprises a base lens formed with a plurality of positive diffractive zones, a plurality of negative diffractive zones, and a step separating a plane where one of the positive diffractive zones starts from a plane at a negative diffractive zone adjacent to the one of the positive diffractive zones ends. In some embodiments of the present invention each diffractive subzone has a constant-sign curvature along a radial direction of the base lens.

According to some embodiments of the invention the base lens is also formed with a plurality of refractive zones.

According to some embodiments of the invention a base power of all diffractive zones is the same, and wherein a power of all refractive zones is different from the base power of all the diffractive zones, thereby providing one or more additional foci.

According to some embodiments of the invention the refractive zones are offset from the base lens to compensate a phase difference induced by light diffracted from the diffractive zones and refracted from the refractive zones.

According to an aspect of some embodiments of the present invention there is provided a lens device. The lens device comprises a base lens formed with: a central zone configured as a negative diffractive zone, a plurality of negative diffractive peripheral zones adjacent to one another extending from a periphery of the base lens towards the central zone, and a plurality of intermediate diffractive zones between the central zone and the negative diffractive peripheral zones. In some embodiments of the present invention each intermediate diffractive zone comprises a positive diffractive subzone and a negative diffractive subzone, wherein the negative and the positive diffractive subzones are arranged alternately, and wherein each diffractive subzone has a constant-sign curvature along a radial direction of the base lens.

According to some embodiments of the invention at least one of the negative and the positive diffractive subzones are characterized by a sawtooth profile.

According to some embodiments of the invention the base lens is devoid of refractive zones.

According to some embodiments of the invention a base power of all positive diffractive subzones is different compared to a base power of all negative diffractive subzones.

According to some embodiments of the invention the device comprises at least two radially separated segments of a non-diffractive region.

According to some embodiments of the invention each of the positive diffractive subzone starts from a plane at which its adjacent negative diffractive subzones ends.

According to some embodiments of the invention an optical surface of the device is devoid of any vertical or substantially vertical steps.

According to an aspect of some embodiments of the present invention there is provided a method of forming a lens. The method comprises forming a diffraction profile on a base lens, wherein the diffraction profile as delineated above and/or detailed hereinbelow.

According to an aspect of some embodiments of the present invention there is provided a method of treating vision of a subject. The method comprises implanting in an eye of the subject a lens device, wherein the lens device is as delineated above and/or detailed hereinbelow.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are graphical representations of a stepped diffractive profile (FIG. 1A), and a sinusoidal diffractive profile (FIG. 1B);

FIG. 2A is an isometric view of a lens device, in accordance with an embodiment;

FIG. 2B is a top view of the lens device of FIG. 2A, in accordance with an embodiment;

FIG. 2C is a sectional view of the lens device along a section plane AA of FIG. 2B, illustrating plurality of diffractive subzones 220a -220d and refractive zones 230a and 230b in accordance with an embodiment;

FIG. 2D is a graphical representation of the lens device of FIG. 2C illustrating plurality of diffractive subzones 220a -220d and refractive zones 230a, 230b, in accordance with an embodiment;

FIG. 2E is a graphical representation of the lens device with refractive profiles 240a and 240b at an offset from the base in accordance with an embodiment;

FIG. 2F is a sectional view of the lens device illustrating plurality of diffractive zones 220a′, 220b′ of different height, in accordance with another embodiment;

FIG. 2G is sectional view of the lens device, illustrating outer periphery 218 and inner periphery 222 of the diffractive subzones 220a -220d, in accordance with an embodiment;

FIG. 2H is sectional view of the lens device illustrating plurality of diffractive zones 242a, 242b, in accordance with another embodiment;

FIG. 2I is a graphical representation of the lens device of FIG. 2H illustrating plurality of diffractive zones 242a, 242b, in accordance with the embodiment;

FIG. 2J is the graphical representation of another embodiment where refractive zones are replaced by diffractive zones (446, 450a, 450b);

FIG. 2K is the normalized irradiance profile of the embodiment depicted in FIG. 2J, in accordance with the embodiment;

FIG. 3 is the sectional view of the lens device illustrating simplified light distribution of the lens device, in accordance with an embodiment;

FIG. 4A is a sectional view of the lens device illustrating the diffractive subzones 400a -400h in accordance with yet another embodiment;

FIG. 4B is a graphical representation of the lens device of FIG. 4A illustrating the diffractive subzones 400a-400h, in accordance with the embodiment;

FIG. 5A is a graphical representation of the lens device illustrating a step 502 between the diffractive zones 220, in accordance with an embodiment;

FIG. 5B is a graphical representation of the lens device illustrating a step 502a and 502b between the diffractive subzones 220, in accordance with an embodiment;

FIG. 5C is a graphical representation of the lens device illustrating negative diffractive sub-zones replaced by refractive subzones in accordance with an embodiment;

FIG. 6A is a perspective view of an exemplary lens device illustrating diffractive zones/subzones 608 on full surface, in accordance with an embodiment;

FIG. 6B is a perspective view of an exemplary lens device illustrating the diffractive portion 602 separated radially from a non-diffractive portion 604, in accordance with an embodiment;

FIG. 7 is a schematic illustration showing a representative example of a diffraction profile suitable for some embodiments of the present invention; and

FIG. 8 is a schematic illustration of a lens device in embodiments in which the lens device comprises haptic structures coupled to the base lens.

### DESCRIPTION OF SPECIFIC EMBODIMENTS OF THE INVENTION

The present invention, in some embodiments thereof, relates to optics and, more particularly, but not exclusively, to a lens providing both positive and negative diffraction.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

When a ray of light moving in air and striking a surface of a light-transmissive substance at an angle α₁ as measured from a normal to the surface, it is refracted into the substance at an angle which is determined by Snell’s law, which is mathematically realized through the following equation:

$${\text{n}}_{\text{A}} sin {α}_{1} = {\text{n}}_{\text{S}} sin {α}_{2}$$

where n_{S} is the index of refraction of the substance, n_{A} is the index of refraction of the air, and α₂ is the angle in which the ray is refracted into the substance. Similarly to α₁, α₂ is measured from a normal to the surface. A typical value of n_{A} is about 1.

As used herein, the term “about” refers to ±10 %.

Another optical phenomenon is diffraction which is the slight bending of light as it passes around the edge of an object, or at an opening thereof. The amount of bending depends on the size of the wavelength of light compared to the size of the opening or edge. If the opening is much larger than the light’s wavelength, the bending will be almost unnoticeable. However, if the two are closer in size or equal, the amount of bending is considerable, and easily seen with the naked eye.

Optical effects resulting from diffraction are produced through the interaction of light waves originating from different regions of the opening causing the diffraction. Illustratively, one can view this interaction as one of two types of interferences: (i) a constructive interference when the crests of two waves combine to produce an amplified wave; and (ii) a destructive interference when a crest of one wave and a trough of another wave combine, thus canceling each other.

The present Inventors exploit the refraction and diffraction phenomena for devising a lens device, optionally and preferably a multifocal lens device. Typically, the lens device of the present embodiments has diffractive power for enabling at least near vision and refractive power for enabling far vision. In some exemplary embodiments of the invention the diffractive power of the lens device also enables intermediate vision, and in some exemplary embodiments of the invention the diffractive power of the lens device enables far vision, intermediate vision, and near vision. The present embodiments also contemplate configurations in which the lens device is substantially devoid of refractive power (e.g., refractive power of less than 0.25 D or less than 0.1 D).

The terms “refractive power” and “diffractive power” as used herein with respect to a particular optical element (either a section of the body of the lens or the lens body as a whole), refer to the dominant optical power of that element. Specifically, a particular optical element is said to have a refractive power if at this element the refractive power dominates the diffractive power, and particular optical element is said to have a diffractive power if at this element the diffractive power dominates the refractive power. If the refractive and diffractive powers are comparable, the element is said to have both optical powers.

The lens device of the present embodiments optionally and preferably comprises a diffraction profile selected so as to provide far focus power X where X is any power from +0 D to about +3.5 D. In some embodiments of the invention the diffraction profile is selected to provide add-power Y, where Y is any power from about +2 D to about +4 D.

The term “add-power” is well known to those skilled in the art of multifocal lenses. To the extent that any further explanation may be required, add-power refers to the amount of optical power difference between the far focus power and the near-focus power.

Preferably, X and/or Y are integers or half-integers (e.g., X = 0D, 0.5 D, 1 D, ..., and/or Y=2 D, 2.5 D, 3D, ...).

In embodiments in which the lens has a refractive power, the refractive power is optionally and preferably substantially uniform across the optical surface of the lens, e.g., with deviations of less than 10% or less than 5%. However, this need not necessarily be the case, since, for some applications, it may not be necessary for the refractive power to be uniform.

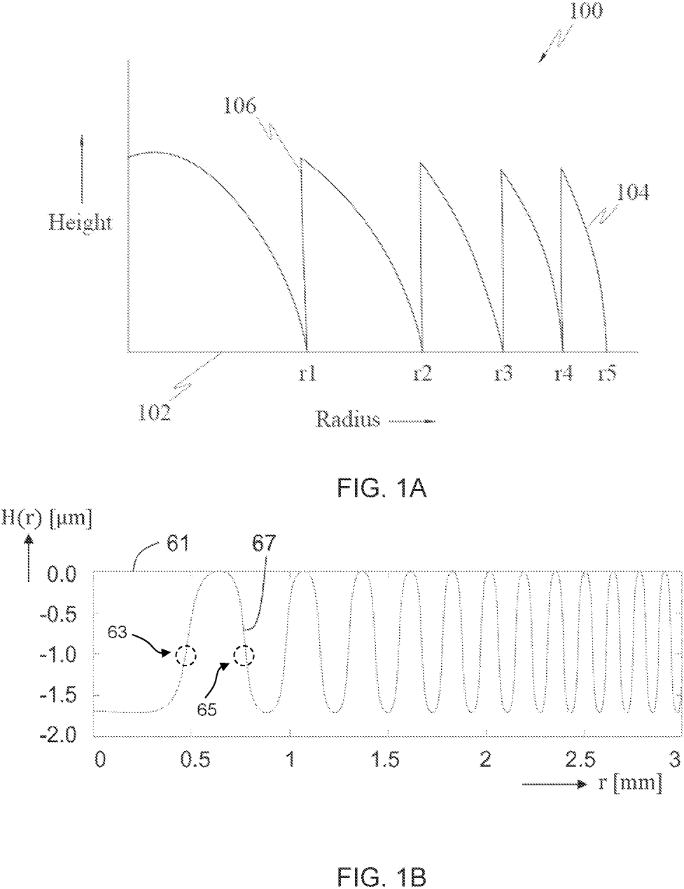

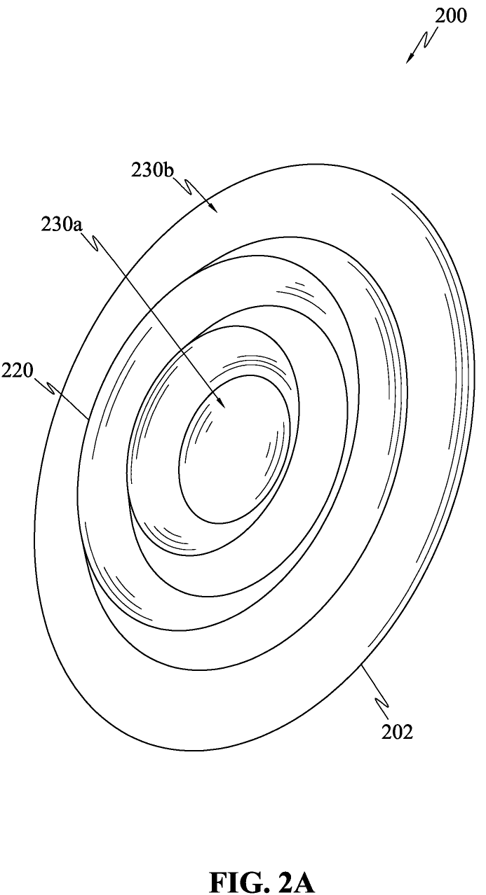

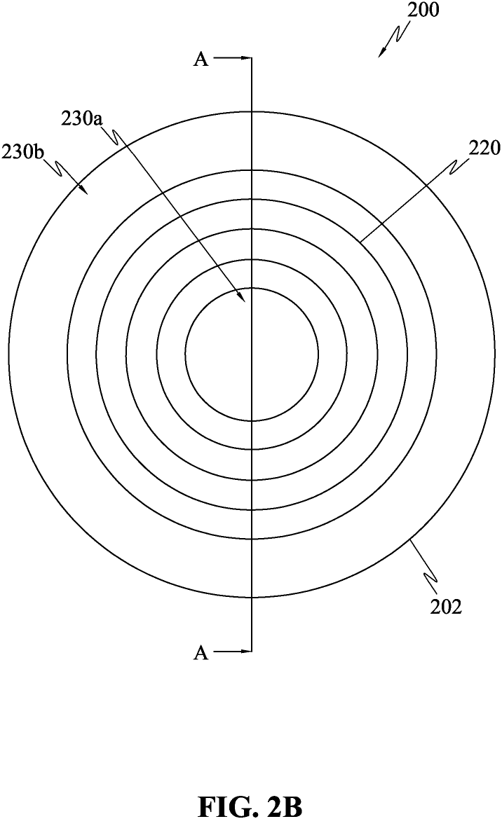

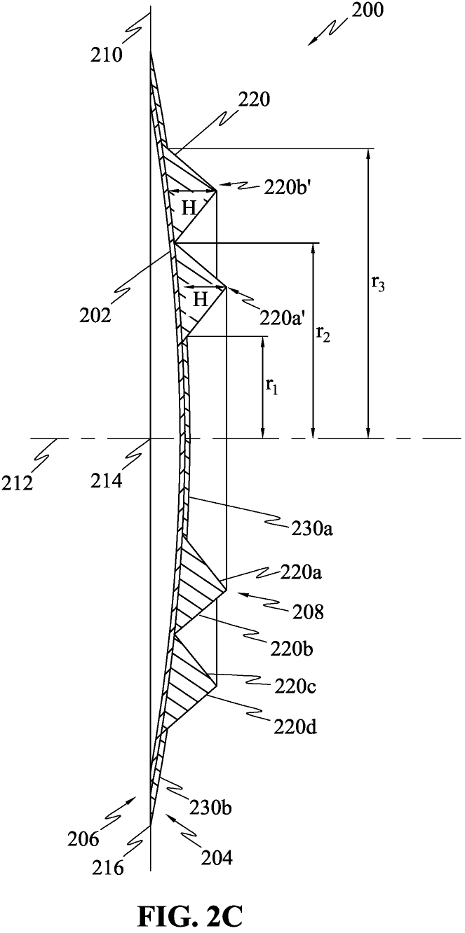

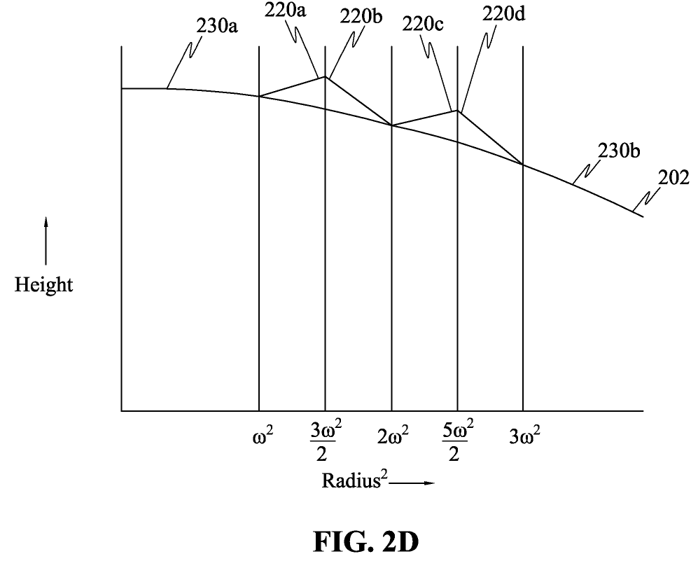

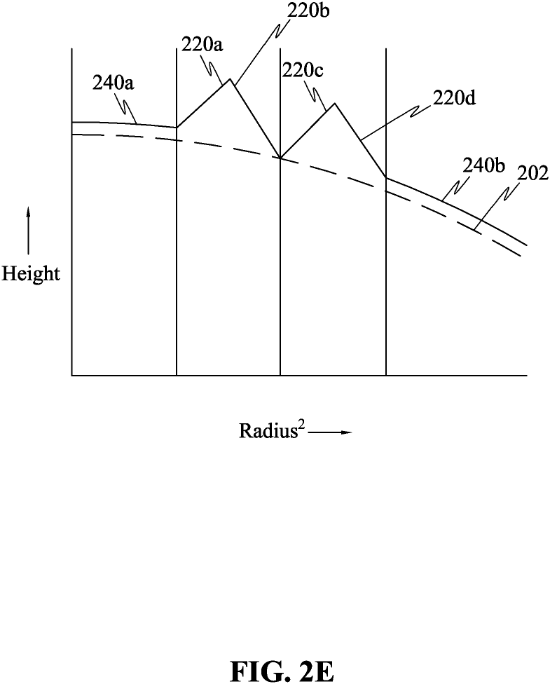

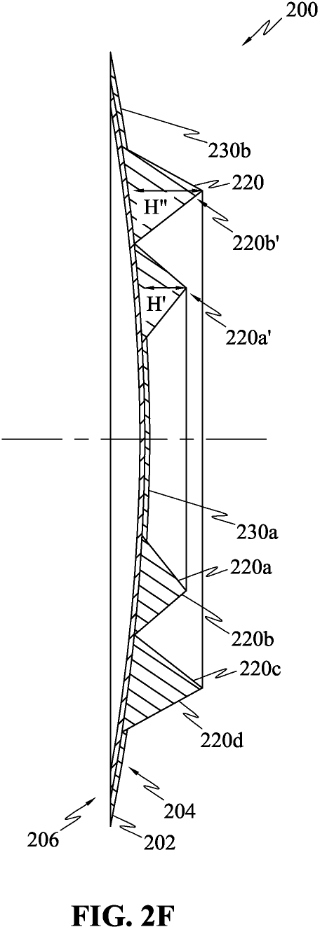

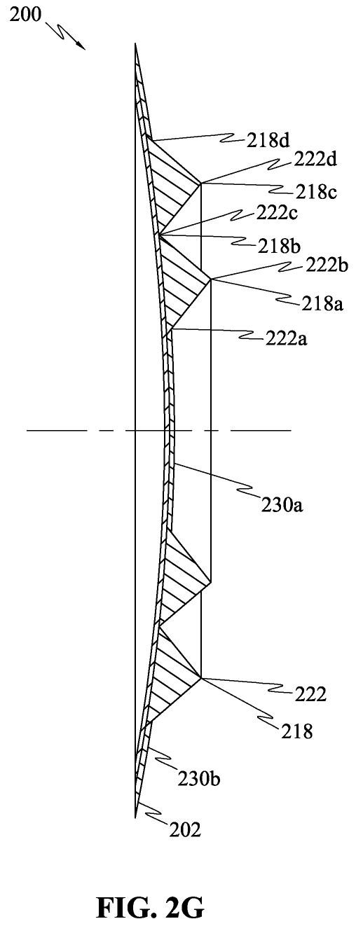

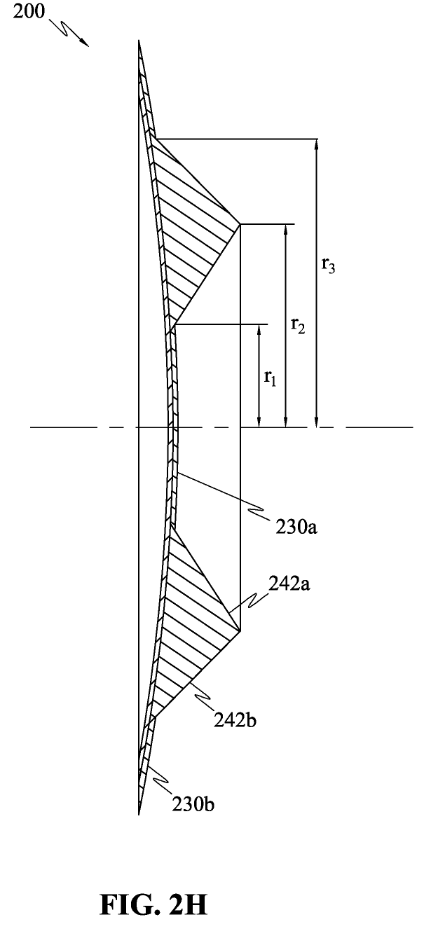

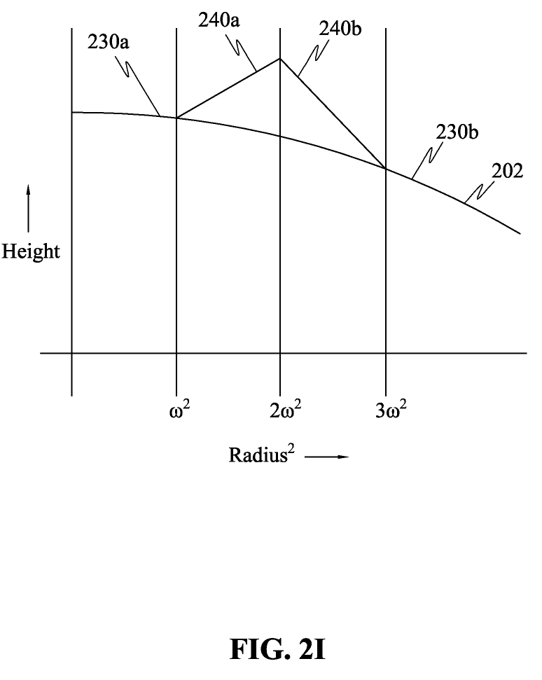

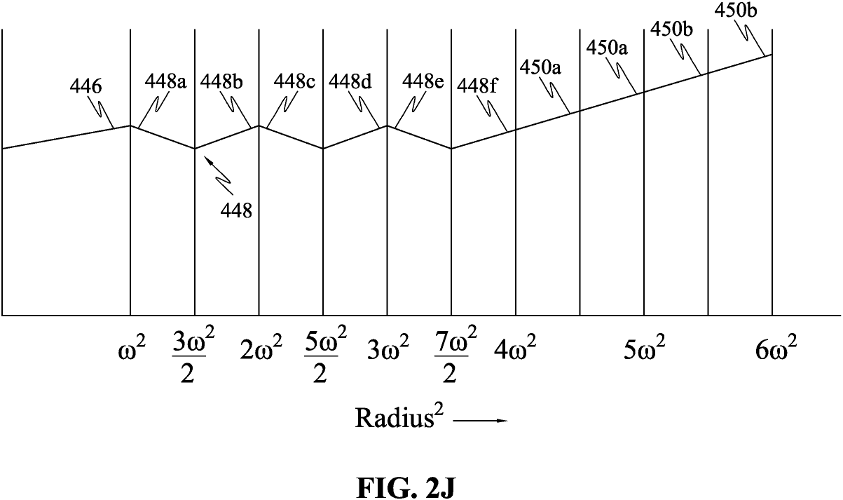

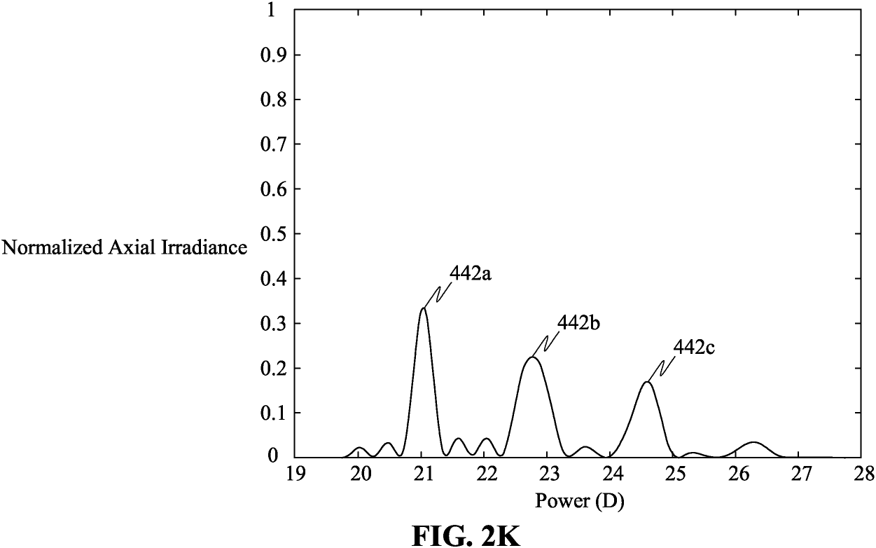

For purposes of better understanding some embodiments of the present invention, as illustrated in FIGS. 2-7 of the drawings, reference is first made to the construction of refractive-diffractive lens as illustrated in FIGS. 1A and 1B.

FIG. 1A illustrates a conventional diffractive refractive lenses 100 with diffractive zones 104. The base of each diffractive zone 104 starts from a base profile 102 of the diffractive refractive lens 100. Consequently, each diffractive zone 104 is demarcated from the adjacent diffractive zone 104 by a vertical step 106. The Inventors found that the presence of the vertical steps 106 between the diffractive zones 104 causes scattering and non-desired dispersion. The scattering and non-desired dispersion at the steps 104, results in considerable loss in image contrast sensitivity because of loss of light.

FIG. 1B illustrates a height profile 67 of another conventional trifocal intraocular ophthalmic lens. The height profile 67 is described by a function H(r) that varies sinusoidally with the square of the radial distance from the center of the lens body. Reference numeral 61 refers to the outer circumference of the front surface of the lens body. Reference numeral 63 exemplifies a point on profile 67 at which the curvature along the radial direction r changes its sign from a positive curvature to a negative curvature, and reference numeral 65 exemplifies a point on profile 67 at which the curvature along the radial direction r changes its sign from a negative curvature to a positive curvature. It is appreciated that points such as 63, 65, exist at each period of profile 67. The Inventor found that the inefficiency of such a profile is far from being satisfactory.

In a search for a diffraction profile that provides a better efficiency, the Inventor devised a lens device which provides alternating positive and negative diffraction. Preferably, the diffraction profile of the lens device has a constant-sign curvature along the radial direction of the lens.

Herein, a constant-sign curvature refers to a curvature that does not change its sign both in zones or sub-zones that provide positive diffraction and in zones or sub-zones that provide negative diffraction.

Mathematically, a curvature can be described by a second derivative of the function that describes the profile as a function of the radial distance from the center of the base lens. Thus, the lens devised by the Inventors has a diffraction profile described by a function for which the sign of the second derivative along the radial direction is the same, both in zones or sub-zones that provide positive diffraction and in zones or sub-zones that provide negative diffraction.

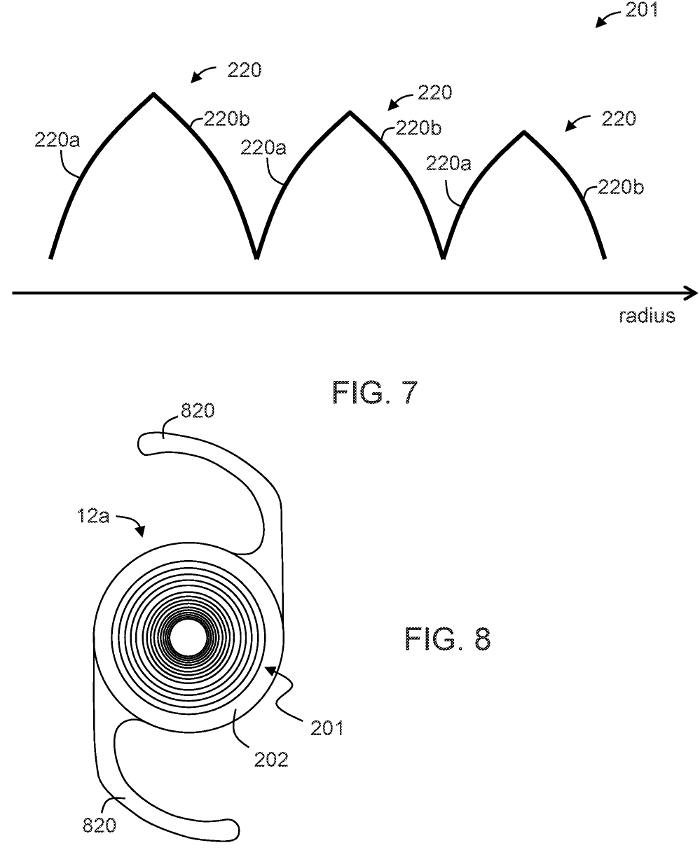

A representative example of a diffraction profile 201 suitable for the present embodiments is illustrated in FIG. 7. The diffraction profile 201 comprises a plurality of zones 220 (three are shown in FIG. 7, but the present disclosure contemplates any number of zones). Each zone 220 include a sub-zone 220a providing positive diffraction, and a sub-zone 220b providing negative diffraction. As shown, profile 220 has a curvature which does not change its sign. In the illustrated embodiment, the curvature is positive throughout sub-zone 220a as well as throughout sub-zone 220b, thus providing higher efficiency compared to conventional sinusoidal profiles, such as the profile shown in FIG. 1B.

Optionally, but not necessarily, device 200 is devoid of vertical or substantially vertical steps across the entire optical region of device 200. The advantage of these embodiments is that the diffractive zones 220 are not demarcated by steps, thus reducing the loss of light and other unwanted dispersion and scattering of light.

Herein, a substantially vertical step is a step which deviate from verticality by a tolerance of less than 10° more preferably less than 5°.

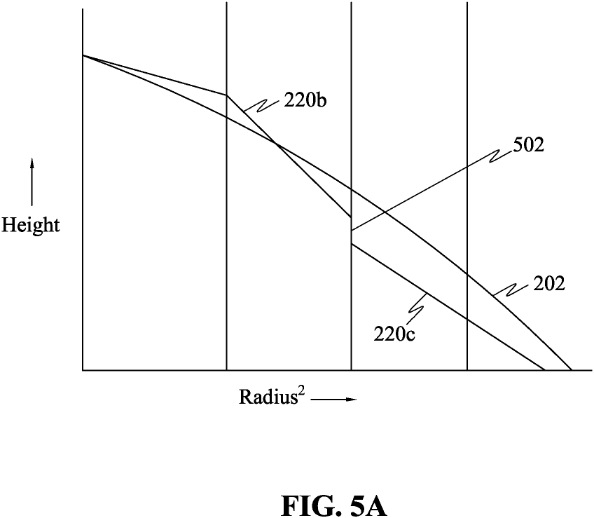

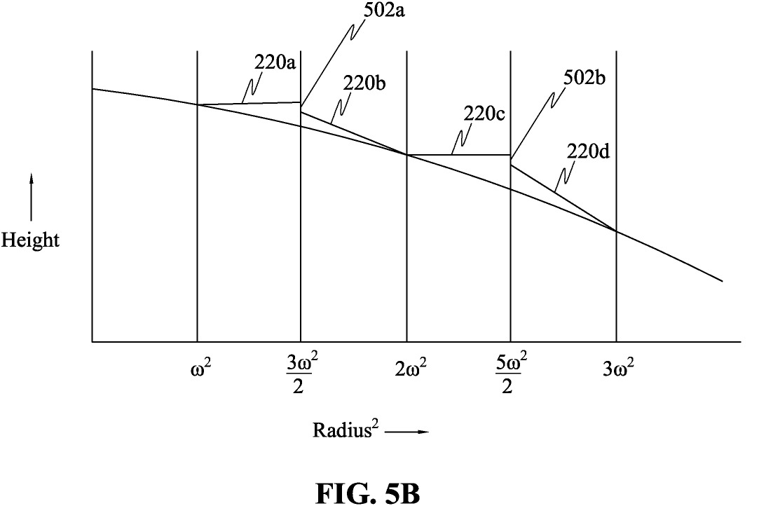

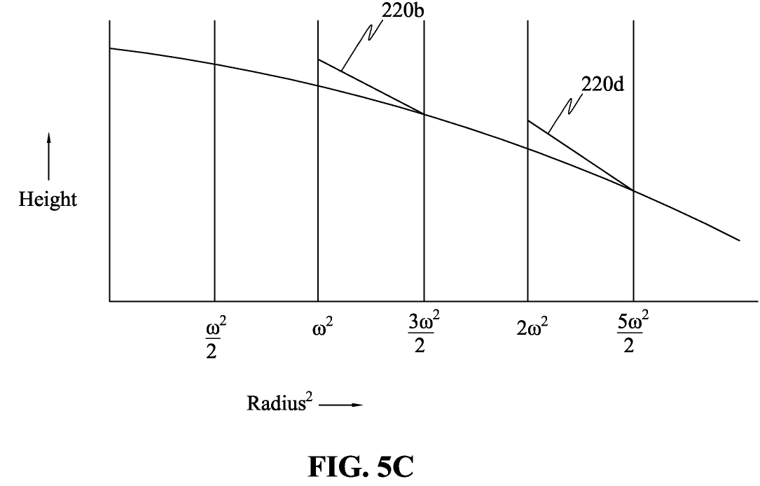

While the embodiments below are described with a particular emphasis to a lens device that is devoid of vertical or substantially vertical steps, it is to be understood that in some embodiments of the present invention the diffraction profile may include one or more steps. These embodiments are described below with reference to FIGS. 5A-C.

FIGS. 2A and 2C illustrate a lens device 200 according to some embodiments of the present invention. In the embodiment illustrated in FIG. 2C, lens device 200 is a diffractive-refractive lens device.

Lens device 200 may be configured to comprise a base lens 202 and a kinoform 208, wherein the lens device 200 may be designed by superimposing the kinoform 208 on the base lens 202.

The term “kinoform” is explained, for example, in “Diffractive Optics-Design, Fabrication and Test” by Donald O’Shea et al., SPIE tutorial texts; TT62 (2004), and refers to diffractive optical elements whose phase-controlling surfaces are smoothly varying. This is different from so-called “binary optical elements” with a discrete number of phase-controlling surfaces.

For simplicity, FIG. 2C shows the kinoform as a non-curved profile. The skilled person would recognize that once the kinoform is superimposed on the base lens, the obtained diffraction profile acquires a curvatures, as illustrated, for example, in FIG. 7.

The lens device 200 has a vertical axis 210 and a horizontal axis 212. The vertical axis 210 and the horizontal axis 212 may intersect at a centre point 214. The base profile 202 may define an anterior surface 204 and a posterior surface 206. The posterior surface 206 and the anterior surface 204 may converge to a converging periphery 216.

The lens device 200 may be used as, but not limited to, a spectacle lens, a contact lens, a phakic lens, an add-on piggyback lens for correction of residual refractive error after intra-ocular lens implantation or as primary intraocular lens during cataract surgery.

The present embodiments contemplate a configuration in which the lens 200 includes toricity, asphericity, or both toricity and asphericity. The toricity and/or asphericity are selected, in some embodiments of the present invention, to correct or mitigate corneal astigmatism and presbyopia. The toricity and asphericity is optionally and preferably on two separate surfaces. However, embodiments in which both toricity and asphericity are present on a single surface are not excluded from the scope of the present invention. A single asphericity may be presented for all cylinder meridians or a variable asphericity may be presented for different meridians. For examples, different degrees of asphericity may be used for the two primary meridians of the astigmatism. Embodiments disclosed herein may be useful for correcting or mitigating other aberrations, such as coma, trefoil, tetrafoil, and the like. Correction of higher order aberrations are also contemplated.

Thus, in some embodiments of the present invention both the anterior surface 204 and the posterior surface 206 are designed using profile of an aspheric lens, in some embodiments of the present invention both the anterior surface 204 and the posterior surface 206 are designed using profile of a toric lens, in some embodiments of the present invention the anterior surface 204 is designed using profile of an aspheric lens and the posterior surface 206 is designed using a profile of a toric lens, in some embodiments of the present invention the anterior surface 204 is designed using profile of a toric lens and the posterior surface 206 is designed using profile of an aspheric lens, in some embodiments of the present invention at least one of the anterior 204 and posterior 206 surfaces is designed using a combination of a profile of a toric lens and a profile of an aspheric lens.

The term “aspheric profile” is well known to those skilled in the art. To the extent that any further explanation may be required, this term is employed herein to refer to a radial profile of a surface that exhibits deviations from a spherical surface. Such deviations can be characterized, for example, as smoothly varying differences between the aspherical profile and a putative spherical profile that substantially coincides with the aspherical profile at the small radial distances from the apex of the profile.

The term “toric profile” is also well known to those skilled in the art. To the extent that any further explanation may be required, this term is employed herein to refer to a radial profile of a surface having a first refracting power along a first meridian and a second refracting power along a second meridian, wherein the first and second meridians are perpendicular to each other and wherein the first and second refracting power differ from each other. Typically, the shape of the respective surface is approximately that of a lateral section of a torus.

The present embodiments also contemplate configurations in which at least one of the anterior 204 and posterior 206 surfaces is designed using a profile of a spherical lens. The present embodiments also contemplate configurations in which a profile of a spherical lens is combined with one or more other profiles (e.g., a profile of a toric lens and/or a profile of an aspheric lens), for example, one of anterior 204 and posterior 206 surfaces is designed using a profile of a spherical lens, and the other surface is designed using a profile of a toric lens, a profile of an aspheric lens, or a profile combining toric and aspheric properties.

In an embodiment, the kinoform 208 may be superimposed on the anterior surface 204 of the lens device 200.

In another embodiment, the kinoform 208 may be superimposed on the posterior surface 206 of the lens device 200.

In yet another embodiment, the kinoform 208 may be superimposed on both the anterior surface 204 and the posterior surface 206 of the lens device 200.

In an embodiment, referring to FIG. 2C, the lens device 200 may comprise plurality of concentric zones, wherein, the plurality of the concentric zones may be configured to comprise refractive zones 230a, 230b and diffractive zones 220. A central zone of the plurality of the concentric zones may be the refractive zone 230a, surrounded by one or more diffractive zones 220a',220b' and the peripheral zone may be the refractive zone 230b. That is to say, the first zone may be the refractive zone 230a, the second and the third zones may be the diffractive zones 220a', 220b' and the peripheral zone may be the refractive zone 230b. Refractive zones 230a and diffractive zones 220 may have radius of curvature r. That is to say, the central refractive zone 230a may have the radius r₁, the first diffractive zone 220a' may have the radius r₂, the second diffractive zone 220b' may have the radius r₃ and so on. The radius r of each zone may be calculated from one of the following equations, well known in the art;

$${r}_{i}^{2} = \frac{2 i λ}{D}$$

wherein, rᵢ is the radius of ith zone, i is the zone number first zone being 1, λ is a representative wavelength in the visible range, and D is the positive or negative add power being provided by the diffractive zone.

Or with following equation

$${r}_{i}^{2} = \frac{\left(2 i + 1\right) λ}{D}$$

wherein, rᵢ is the radius of ith zone, i is the zone number first zone being 0, λ is a representative wavelength in the visible range, D is the positive or negative add power being provided by the diffractive zone 220.

Or with following equation;

$${r}_{i}^{2} = {r}_{0}^{2} + \frac{2 i λ}{D}$$

wherein, rᵢ is the radius of ith zone, i is the zone number first zone being 0, λ is a representative wavelength in the visible range, r₀ is the radius of the center zone, and D is the positive or negative add power being provided by the diffractive zone.

In the present embodiments, the kinoform is a combination of a positive saw-tooth shape and a negative saw-tooth shape which are interlaced thereamongst to form an alternating pattern of negative and positive diffraction surfaces.

Referring to FIG. 2C and FIG. 2D, each of the diffractive zones 220a', 220b' may be configured to comprise a pair of subzones of positive and of negative diffractive profiles. That is to say, the first diffractive zone 220a' may be configured to comprise negative diffractive subzone 220a and positive diffractive subzone 220b, the second diffractive zone 220b' may be configured to comprise negative diffractive subzone 220c and positive diffractive subzone 220d and so on. The plurality of diffractive zones 220a', 220b' may have a height H.

In an embodiment, the radius of curvature along axis 212 of the central refractive zone 230a and the peripheral refractive zone 230b (hence the optical power) may be equal to the base power of diffractive zone 220.

In another embodiment, the radius of curvature along axis 212 of the central refractive zone 230a and peripheral refractive zone 230b (hence the optical power) may be different as compared to base power of diffractive zone 220.

In an embodiment, referring to FIG. 2C, the height (from the base profile 204) of the plurality of the diffractive zones 220 may be the same. That is to say, the height H of the diffractive zone 220a' may be same as the height H of the diffractive zone 220b’ and so on.

Reference is now made to FIG. 2F, which is a schematic illustration of lens device 200 in embodiments in which the heights (from the base profile 204) of the diffractive zones are different. In the embodiment illustrated in FIG. 2F, lens device 200 is a diffractive-refractive lens device.

As an example, the height of the first diffractive zone 220a' (the first diffractive zone 220a' may comprise the negative diffractive subzone 220a and the positive diffractive subzone 220b) may be different as compared to height of the second diffractive zone 220b′ (the second diffractive zone 220b' may comprise the negative diffractive subzone 220c and the positive diffractive subzone 220d). The height of the first diffractive zone 220a' H’ may be smaller than the height of the second diffractive zone 220b' H” (H’ < H”).

FIG. 2D is a graphical representation of the height H and the radius of the diffractive subzones 220a, 220b and 220c and 220d of the lens device 200. The x-axis of the graph represents the radius squared denoted ω² of the diffractive zones/subzones 220 and refractive zones 230a, 230b and the y-axis of the graph represents the height H of the diffractive subzones 220 and refractive zones 230a, 230b. The radius r₁ of the central refractive zone 230a may be r₁ = ω, the radius r2 of the negative diffractive subzone 220a may be

$${\text{r}}_{2} = \sqrt{\frac{3 {ω}^{2}}{2}} ,$$

the radius r₃ of the positive diffractive subzone 220b may be

$${\text{r}}_{3} = \sqrt{2 {ω}^{2}}$$

and so on. The height h of the diffractive subzones 220a may be the multiple of H, multiple being an integer and H calculated from the following equation, well known in the art;

$$H = \frac{λ}{{n}_{1} - {n}_{0}}$$

wherein, λ is a representative wavelength in the visible range, n₁ is the refractive index of the lens 200, and n₀ is the refractive index of aqueous humour.

In another embodiment, referring to FIG. 2E, the wave front of the light diffracted from the diffractive zones 220 and the wave front of the light refracted from refractive zones 240a and 240b may have a phase difference. To compensate the phase difference, the refractive zones 240a and 240b may be offset from the base profile 202 along the horizontal axis (not shown, see axis 212 in FIG. 2C). The offset is optionally and preferably selected such that the wave front of the light diffracted from the diffractive zones 220 and the wave front of the light refracted from the refractive zones 240a and 240b are in phase. This configuration thus compensates the phase difference between the refracted wave front and the diffracted wave front.

Reference is now made to FIG. 2G, which is a schematic illustration of lens device 200 in embodiments in which each of the plurality of the diffractive subzones 202 further defines an outer periphery 218 and an inner periphery 222. In the embodiment illustrated in FIG. 2G, lens device 200 is a diffractive-refractive lens device.

As an example, the first diffractive subzone 220a may define an outer periphery 218a and an inner periphery 222a, the second diffractive subzone 220b may define an outer periphery 218b and an inner periphery 222b, the third diffractive subzone 220c may define an outer periphery 218c and an inner periphery 222c and so on.

In the configuration shown in FIGS. 2C, 2F and 2G, the kinoform 208 is configured to comprise alternating negative and positive diffractive subzones/zones, and the plurality of diffractive zones/subzones 220 are not demarcated by vertical steps. That is to say, the diffractive subzones 220 starts from the same plane at which the adjacent diffractive subzone 220 ends, without presence of any vertical step in between them. As an example, the inner periphery 222b of the positive diffractive subzone 220b starts from the outer periphery 218a of the negative diffractive subzone 220a, the inner periphery 222c of the negative diffractive zone 220c starts from the outer periphery 218b of the positive diffractive zone 220b and so.

Reference is now made to FIG. 2H, which is a schematic illustration of lens device 200 in embodiments in which the diffractive zones may not be subdivided into subzones. In the embodiment illustrated in FIG. 2H, lens device 200 is a diffractive-refractive lens device. In FIG. 2H, the first diffractive zone may comprise a negative kinoform 242a, and the second diffractive zone may comprise a positive kinoform 242b and so on. As an example, the central zone of the lens device 200 may be the refractive zone 230a, the second zone may be the negative diffractive zone 242a, the third zone may be the positive diffractive zone 242b and the peripheral zone may be the refractive zone 230b. The central refractive zone 230 may have radius r₁, the first negative diffractive zone 242a may have the radius r₂, the second positive diffractive zone 242b may have the radius r₃ and so on.

FIG. 2I is a graphical representation of the embodiment described in FIG. 2H. The x axis represents the radius squared of the refractive zones 230a, 230b and the diffractive zones 240a, 240b and the y axis represents the height of the refractive zones 230a, 230b and the diffractive zones 240a, 240b The radius r₁ of the first refractive zone 230a may be ω, the radius r2 of the first diffractive zone 242a may be

$$\sqrt{2 {ω}^{2}}$$

and the radius r₃ of the second diffractive zone 242b may be

$$\sqrt{3 {ω}^{2}}$$

and so on.

In yet another embodiment, referring to FIG. 2J, the centre zone (446) and the peripheral zone (450a, 450b) may be configured to comprise negative diffractive subzones. That is to say, the central zone, first zone, second zone and so on and the peripheral zone comprises of only diffractive profiles. Further, the second zone (448), the third zone (448) and the fourth zone (448) may be configured to comprise alternating positive diffractive and negative diffractive subzones. That is to say, the second zone (448) may comprise of a positive diffractive subzone (448a) and a negative diffractive sub zone (448b), the third zone (448) may comprise of a positive diffractive subzone (448c) and a negative diffractive subzone (448d) and so on. The negative diffractive centre zone (446) and the negative diffractive peripheral zone (450a, 450b) may result in increasing the light distribution at order -1 which represents the distance focus.

FIG. 2K is the normalized irradiance profile of the profile graphically represented in FIG. 2J with a pupil diameter of 3.2 mm. Referring to FIG. 2K, a first peak (highest peak) 424a, a second peak 424b and a third peak 424c represents the distance focus, intermediate focus and the near focus, respectively.

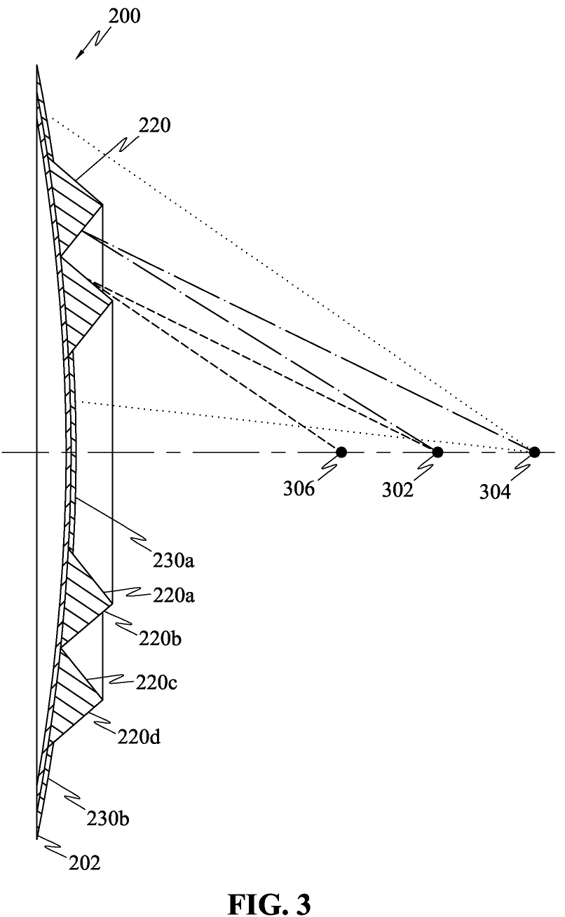

Reference is now made to FIG. 3, which is a schematic illustration of lens device 200 in embodiments in which lens device 200 is a diffractive-refractive lens device that achieves multifocal capability through principles of diffraction and refraction. The refractive zones 230a and 230b may focus light on focal point 304. Positive diffractive subzones may split the light onto two foci 302 and 306. Negative diffractive subzones split the light on two foci 302 and 304.

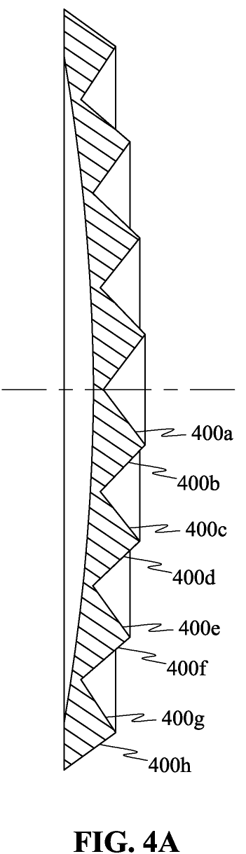

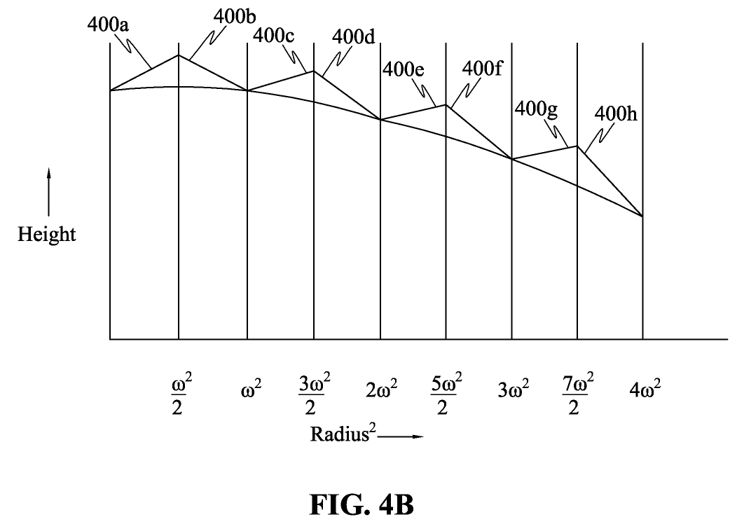

Reference is now made to FIG. 4A, which is a schematic illustration of lens device 200 in embodiments in which lens device 200 is a diffractive lens device that is optionally and preferably substantially devoid of refractive power. These embodiments are similar to the embodiments in which lens device 200 is a diffractive-refractive lens device, except that the refractive zones may be replaced by diffractive zones, so that the whole surface from centre to periphery has diffractive zone/subzones. As an example, the kinoform 208 may be configured to comprise plurality of diffractive zones 400a - 400h.

FIG. 4B discloses the graphical representation of the embodiment described in FIG. 4A. The refractive zones of lens 200 are replaced by alternately configured diffractive subzones 400a-400h from centre to periphery.

The lens device 200 may be used either as a monofocal or a multifocal (bifocal, trifocal etc) lens. When the height of the plurality of diffractive zones 220 is multiple of H and the multiple being an absolute number except 0, then the diffractive region of the lens device 200 may provide a single focus. In such a scenario, the lens device 200 may be used as monofocal lens. As an example, if the base power is 20 D and add power is +2 D (provided by the diffractive zones), then the light may be focused on single focal point with power 22 D.

The lens device 200 may be used a bifocal lens, when the height of the plurality of diffractive zones 220 is same as in previous example but the kinoform 208 may be configured to comprise alternately configured positive diffractive zones and negative diffractive zones. In such a scenario, the light may be focused onto 2 foci; wherein the positive diffractive zones may focus the light onto one focal point and the negative zones may focus the light onto second focal point. As an example, if base power is 20 D and add power is +2 D (add on power provided by the positive diffractive zones) and -2 D (add on power provided by the negative diffractive zones), then all light may focus on the focal points 18 D and 22 D.

The lens device 200 may be used a trifocal lens, when the kinoform may be configured to comprise alternately configured positive diffractive zones and negative diffractive zones, wherein the plurality of the positive diffractive zones and the plurality of negative diffractive zones may be configured with height h being an integer which is not whole number . In such a scenario, the light may be focused onto 3 foci; wherein the positive diffractive zones may focus the light onto first and second focal points and the negative zones may focus the light onto second and third focal points. As an example, if the base power is 20 D and h = 0.5 H, then one fourth light will be focussed on 18 D, half of light will be focussed on 20 D and one fourth light will be focussed on 22 D.

The lens device 200 may be used a quadrifocal lens, when the base power of all positive subzones/zones may be different to the base power of all negative subzones/zones. For example, if all negative diffractive zones/subzones have base power of 20 D and add power of -1.5 D and all positive zones/subzones have base power of 19 D and add power of +1.5 D, light will be focussed on four foci; 18.5 D, 19 D, 20 D and 20.5 D. The refractive zones may have a power which can augment light on any one of the foci formed by diffractive region.

In an embodiment, the refractive power of the central refractive zones 230a and the peripheral refractive zone 230b may be same as the refractive power of the base lens 202 of the diffractive-sub zones 220a-220d.

In an embodiment, power of all refractive subzones may be same.

In an embodiment, power of all refractive subzones may be different.

In an embodiment, the power of the refractive zones 230a and 230b and refractive power of base curve 204 of the diffractive-sub zones 220a-220d may be same.

In an embodiment, the power of the refractive zones 230a and 230b and refractive power of base curve 204 of the diffractive-sub zones 220a-220d may be different.

In an embodiment, focusing power of the plurality of diffractive positive zones/sub-zones 220 may be same as focusing power of the plurality of diffractive negative zones/sub-zones 220. That is to say, the add-on power provided by the positive diffractive zone/sub-zones 220 may be same as the add-on power provide by the negative diffractive zone/sub-zones 220. As an example, the positive diffractive zone/sub-zone 220 may provide add on power +2 and the negative diffractive zone/sub-zone 220 may provide add-on power -2.

In another embodiment, focusing power of the plurality of diffractive positive zones/sub-zones 220 may be different as compared to the focusing power of the plurality of diffractive negative zones/sub-zones 220. That is to say, the add-on power provided by the positive diffractive zone/sub-zones 220 may be different as compared to the add-on power provide by the negative diffractive zone/sub-zones 220. As an example, the positive diffractive zone/sub-zone 220 may provide add on power +2 and the negative diffractive zone/sub-zone 220 may provide add-on power -1. In such a scenario for compensating the unequal power, a step may be provided between the plurality of diffractive zones 220. Referring to FIG. 5A, step 502 may be introduced between the second zone 220b and the third zone 220c for achieving equal power distribution. The step 502 may be introduced between the plurality of diffractive zones 220, wherein the outer periphery 218 or the inner periphery 222 of the diffractive zones 220 may be at a height H’ from the base profile 202 and quantum of height H’ is multiple of H. That is to say, H’ = aH, wherein ‘a’ is an integer.

In another embodiment, referring to FIG. 5B the height of the negative and positive diffractive subzones/zones may be different. As an example, the height of the negative diffractive zones 220a, 220c may be different as compared to the height of the positive diffractive zones 229b, 220d. In such a scenario, step may be configured at the junction of negative and positive diffractive subzone/zone. That is to say, a step 502a may be introduced between the negative diffractive zone 220a and the positive diffractive zone 220b and a step 502b may be introduced between the negative diffractive zone 220c and the positive diffractive zone 220d.

In another embodiment, referring to FIG. 5C, the negative diffractive subzones/zones 220a, 220c may be replaced by refractive subzones/zones.

In yet another embodiment, the positive diffractive subzones/zones 220b, 220d may be replaced by refractive subzones/zones.

In another embodiment one or more of diffractive subzones be replaced by refractive subzones/zones.

In another embodiment one or more of diffractive zones may be replaced by multi order diffractive zones/subzones.

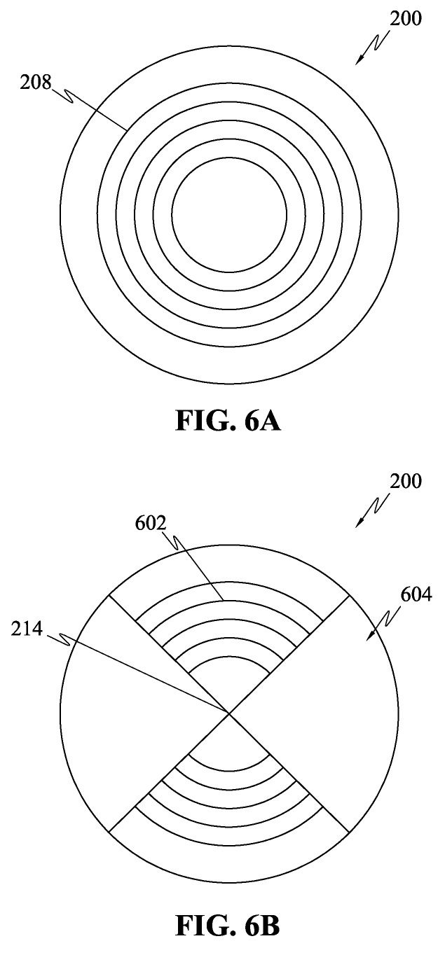

FIG. 6A discloses an embodiment wherein the lens device 200 may be constructed by having diffractive profile 208 onto the complete surface (radially 360 degrees) of the base lens 202. FIG. 6B discloses an embodiment wherein the lens device 200 may be constructed by having a diffractive profile onto a portion of the surface of the base lens 202. The base lens 202 of the lens device 200 may have some portion diffractive 602 that may define the kinoform superimposed on the base lens 202 and some non-diffractive portion 604 wherein the non-diffractive portion 604 is the base lens 202 with no kinoform superimposed. The kinoform of portion 602 may be superimposed radially from the centre point 214 towards the converging point 216. The kinoform of portion 602 may be the same, mutatis mutandis, as kinoform 208 described above.

In an embodiment, the lens device 200 may be used as either a monofocal lens or a multifocal lens by varying the height H of the plurality of the diffractive zones. The lens device 200 may achieve monofocal capability or multifocal capability through the principles of diffraction and refraction. The lens device 200 may provide focus for near vision, far vision and intermediate vision by having three (trifocal) points. The three focal points may be provided by refraction from the base lens 202, diffraction from the plurality of positive diffractive zone 220B and diffraction from the plurality of negative diffractive zones 220a, 220c.

The intensity of the light falling on each of the foci may be varied by changing the height of the plurality of the diffractive zones 220.

Reference is now made to FIG. 8 which is a schematic illustration of lens device 200 in embodiments in which device 200 comprises haptic structures 820 coupled to the base lens 202. These embodiments are particularly useful when device 200 is used as an intraocular lens device, in which case haptic structures 820 can be used for placing and optionally anchoring device 200 into the eye of the subject (e.g., in the ciliary sulcus or the anterior chamber). To allow accurate aligning of lens device 200 during implantation, base lens 202 is optionally and preferably provided with marks (not shown).

The lens device of the present embodiments can be fabricated in any technique known in the art. Generally, the present embodiments form on a substance a plurality of concentric annular zones separated by slanted steps, wherein the concentric zones effect both diffraction and refraction of incident light, while the steps are substantially devoid of any diffractive or refractive power. The substance on which the zones and steps are formed can be an unprocessed or partially processed lens body, in which case the formation of zones and steps serves for forming the lens device directly. Alternatively, the substance can be a mold, in which case the formation of zones and steps serves for forming a lens mold for mass fabrication of lens devices. In these embodiments, the lens device can be cased using the lens mold, as known in the art.

The formation of zones and steps may be done by any convenient manufacturing means, including, for example, a computer-controllable manufacturing device, molding or the like.

A “computer controllable manufacturing device” refers to a device that can be controlled by a computer system and that is capable of producing directly a lens body or a mold for producing a lens device. Any known, suitable computer controllable manufacturing device can be used in the invention. Exemplary computer controllable manufacturing devices includes, but are not limited to, lathes, grinding and milling machines, molding equipment, and lasers. In various exemplary embodiments of the invention a Computerized Numeric Controlled (CNC) lathe machine is used, such as the lathers marketed under the trade names DAC™ Vision, Optoform and CareTec.

A Fast Tool Servo (FST) is optionally employed to form the toric profile. In these embodiments, the lathe machine can generate the diffractive pattern and the aspheric profile, and the FTS can generate the toric profile.

The present embodiments also contemplate a method of treating vision of a subject in need thereof. The method comprises implanting a multifocal lens device in an eye of the subject, thereby treating the vision of the subject. The multifocal lens device preferably comprises one of the lenses device detailed hereinabove. The method can be executed, for example, while or subsequently to a cataract surgery.

As used herein the term “about” refers to ± 10 %.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A lens device, comprising a base lens formed with a plurality of diffractive zones, each having a negative diffractive subzone and a positive diffractive subzone, said negative and said positive diffractive subzones being arranged alternately, wherein each diffractive subzone has a constant-sign curvature along a radial direction of said base lens.

2. The device according to claim 1, wherein at least one of said negative and said positive diffractive subzones are characterized by a sawtooth profile.

3. The device according to claim 1 , wherein said base lens is also formed with a plurality of refractive zones.

4. The device according to claim 3, wherein a base power of all diffractive zones is the same, and wherein a power of all refractive zones is different from the base power of all the diffractive zones, thereby providing one or more additional foci.

5. The device according to claim 3, wherein said refractive zones are offset from said base lens to compensate a phase difference induced by light diffracted from said diffractive zones and refracted from said refractive zones.

6-10. (canceled)

11. The device according to claim 1, comprising at least two radially separated segments of a non-diffractive region.

12. (canceled)

13. The device according to claim 1, wherein each of the positive diffractive subzone starts from a plane at which its adjacent negative diffractive subzones ends.

14. (canceled)

15. The device according to claim 1, wherein an optical surface of the device is devoid of any vertical or substantially vertical steps.

16. (canceled)

17. The device according to claim 1, wherein at least one diffractive zone comprises a step between a respective negative diffractive subzone and a respective positive diffractive subzone, said step separating a plane where said respective positive diffractive subzone starts from a plane at which said respective negative diffractive subzone ends.

18. (canceled)

19. A lens device, comprising a base lens formed with a plurality of positive diffractive zones, a plurality of negative diffractive zones, and a step separating a plane where one of said positive diffractive zones starts from a plane at a negative diffractive zone adjacent to said one of said positive diffractive zones ends, wherein each diffractive zone has a constant-sign curvature along a radial direction of said base lens.

20. The device according to claim 19, wherein at least one of said negative and said positive diffractive zones are characterized by a sawtooth profile.

21. The device according to claim 19 , wherein said base lens is also formed with a plurality of refractive zones.

22-34. (canceled)

35. The device according to claim 1, comprising a central zone configured as a negative diffractive zone; and
a negative diffractive peripheral zone at a periphery of said base lens.
